(19) United States
(12) Patent Application Publication
(10) Pub. No.: US 2009/0060844 A1
(43) Pub. Date: Mar. 5, 2009

(54) METHOD AND CONTRAST AGENT FOR IMAGING A PROSTATE TUMOR

(76) Inventors: Jens Fehre, Hausen (DE); Ralf Nanke, Neunkirchen am Brand (DE); Martin Stetter, Muenchen (DE)

Correspondence Address: SCHIFF HARDIN, LLP;PATENT DEPARTMENT, 6600 SEARS TOWER, CHICAGO, IL, 60606-6473, US

(21) Appl. No.: 12/203,201

(22) Filed: Sep. 3, 2008

(30) Foreign Application Priority Data
Sep. 3, 2007 (DE) 10 2007 041 836.3

(51) Int. Cl. A61K 49/18

(52) U.S. Cl. 424/9.32

(57) ABSTRACT

In a method for imaging a prostate tumor in which a contrast agent that can be distributed via the circulatory system is administered to the patient, the contrast agent contains at least one type of biomarkers which are bound to a ferromagnetic particle and specifically bind to a target molecule typical of the tumor and formed within the tumor or in a tissue adjacent to the tumor. The imaging ensues with the use of magnetic resonance tomography.

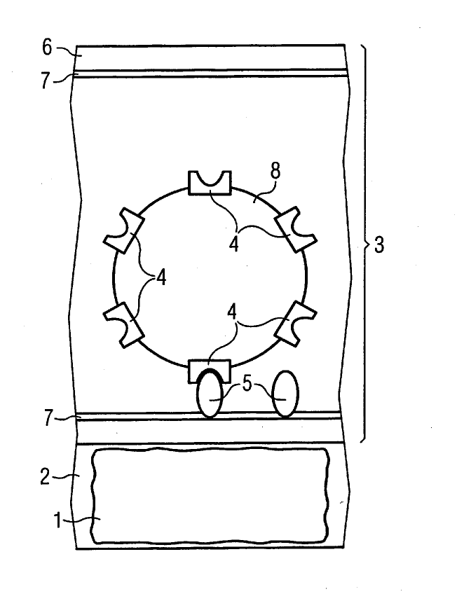

### BACKGROUND OF THE INVENTION

1. Field of the Invention

The invention concerns a method for imaging a prostate tumor and a contrast agent suitable for this purpose.

2. Description of the Prior Art

Obviously it is desirable to detect or diagnose a prostate tumor as unambiguously as possible, in particular when it is still in the early stage. The standard method for diagnosing prostate cancer is to measure concentration of prostate-specific antigen (PSA) and palpation as well as, given suspicion, implementation of biopsies to extract tissue samples. Such a procedure is less selective and cannot differentiate prostate cancer well from prostatitis or BPH. Under the circumstances, this requires multiple biopsies that are unpleasant for the patient. A series of alternative methods are applied in addition to the cited standard method. Points of high blood vessel density can be identified with contrast-enhanced ultrasound examination, possibly to guide biopsies. However, an increased blood vessel density is not specific for a prostate tumor since it also occurs in the case of prostatitis. A molecularly targeted, contrast-enhanced ultrasound examination is generally considered for imaging or prostate tumors or cancerous tumors. Although this is cost-effective, it is not high-resolution, such that it is less suitable for high-quality diagnosis purposes. A further technique is the optical imaging of intrinsic signals such as, for example, the water concentration of a tissue.

### SUMMARY OF THE INVENTION

An object of the invention to provide an additional method for imaging of a prostate tumor and a contrast agent suitable for this purpose.

This object is achieved by a method according to the invention, wherein a contrast agent that can be distributed via the circulatory system is administered to the patient, the contrast agent containing at least one type of biomarkers which bond to a ferromagnetic particle and which specifically bind to a target molecule typical of the tumor and formed within the tumor or in a tissue adjacent to the tumor. The body region of interest of the patient (thus the prostate or a tumor present therein) is imaged with the use of magnetic resonance tomography. Due to the specific biomarker specifically binding in the tumor tissue or to tissue adjacent to this, the contrast agent is taken up nearly exclusively (or at least to an overwhelming degree) in the area or in the tissue that should be subjected to a diagnostic assessment. The ferromagnetic particles (for example containing super-paramagnetic iron oxide) are thus specifically accumulated in the body tissue of interest, thus a prostate tumor. A contrast enhancement is thus specifically achieved essentially only in the tumor tissue, such that this is clearly delimited relative to the healthy tissue surrounding it.

In a preferred embodiment biomarkers are used that bind to a target molecule typical of a specific stage of a prostate tumor. In this way the prostate tumor not only can be imaged but also a conclusion can be made as to the stage of the tumor. For example, CEACAM-1 molecules are expressed in the endothelium of blood vessels of a tissue adjacent to the prostate tumor in the stage of high-grade prostatic intraepithelial neoplasy (hgPIN), a pre-stage of prostate cancer. A biomarker binding to this molecule, primarily a CEACAM-1 antibody, is accordingly used for the contrast agent according to the invention. Biomarkers known as aptamers and spiegelmers can additionally be used. These are short, stable and specifically binding RNA chains, the spiegelmers being the mirror-symmetrical counterparts of the aptamers. Anticalins, which are polypeptide chains composed of approximately 180 amino acids that are easy to produce, are likewise suitable. Anticalins have similar specific binding properties like the antibodies, but are easier to produce than these.

In another embodiment, a later, already-malignant stage of the prostate tumor is targeted, namely one in which compositions known as blood vessel growth factors—for example the molecule VEGF or alpha(v)-beta(3)-integrin—are to be found in the endothelium of the blood vessels of the tumor due to an occurred blood vessel formation. The aforementioned molecules can likewise be used as biomarkers. VEGF or integrin ligands are additionally considered as well. Viruses that are harmless to humans can also be used as biomarkers both in the method targeted at the hgPIN pre-stage and the method targeted at the late stage of the prostate tumor, with which viruses a particle or molecule selectable with the respective imaging method is specifically connected to protein shells binding to the cited target molecules. The viruses can be M13 phages, for example. The specific binding properties of the shell proteins of the viruses can, for instance, be achieved by a targeted biological evolution in which viruses and target molecules are brought into contact in aqueous solution and a selection of binding viruses ensues. The above analogously applies with regard to a contrast agent according to the invention.

### BRIEF DESCRIPTION OF THE DRAWING

The single FIGURE schematically illustrates a prostate exhibiting a tumor and a blood vessel.

### DESCRIPTION OF THE PREFERRED EMBODIMENTS

The FIGURE is a schematic representation of a prostate 2 exhibiting a tumor 1 as well as a blood vessel 3. Blood vessels 3 are either components of the prostate tumor 1 (if this is already in the angiogenesis stage) or are parts of a tissue adjacent to this (in the case of a pre-stage of prostate cancer). In each case, the target molecules 5 identifiable by the contrast agent or the biomarkers 4 contained therein are present in the wall 6 or the endothelium 7 of the blood vessels 3. A biomarker is bound to a ferromagnetic particle 8 containing a paramagnetic material such as iron oxide. In principle it is sufficient when a biomarker 4 is connected with a particle 8. For example, the particles 8 have a diameter of 10 nm-100 nm. In order to achieve a reliable connection to the target molecules 5, it is advantageous for multiple biomarkers 4 to be connected with the particle 8; it is virtually coated with biomarkers 4. It is thereby ensured that binding to a target molecule 5 can ensue in a number of different rotation positions of the particle 8. An uptake in the target tissue (thus in the prostate tumor 1) already occurs after a short period of time after the (for example intravenous) administration of the contrast agent, such that an acquisition with MRT can then occur. If, for example, it is not determined or not certain whether tumor is present in an early or late stage, the contrast agent can contain both biomarkers that bind to target molecules of the early stage (CEACAM-1) or the later stage (for example VEGF or alpha(v)-beta(3)-integrin).

Although modifications and changes may be suggested by those skilled in the art, it is the intention of the inventors to embody within the patent warranted hereon all changes and modifications as reasonably and properly come within the scope of his contribution to the art.

We claim as our invention:

1. Method for imaging a prostate tumor comprising administering a contrast agent to a patient that is distributed via the circulatory system of the patient, and employing a contrast agent that contains at least one type of biomarkers which are bound to a ferromagnetic particle and specifically bind to a target molecule typical of the tumor and formed within the tumor or in a tissue adjacent to the tumor, and imaging the tumor enhanced by the contrast agent by magnetic resonance tomography.

2. A method according to claim 1, comprising employing a contrast agent biomarkers that bind to a target molecule typical of a specific stage of a prostate tumor.

3. A method according to claim 2, comprising employing biomarkers binding to the CEACAM-1 molecule.

4. A method according to claim 3, comprising employing biomarkers selected from the group antibodies, aptamers, spiegelmers, anticalins, viruses.

5. A method according to claim 2, comprising employing biomarkers binding to VEGF.

6. A method according to claim 2, comprising employing biomarkers binding to alpha(v)-beta(3)-integrin.

7. A method according to claim 5 comprising employing a biomarker selected from the group consisting of antibodies, ligands, aptamers, spiegelmers, anticalins, viruses.

8. A contrast agent for imaging a prostate tumor with the use of magnetic resonance tomography, containing at least one type of biomarkers that specifically bind to a target molecule typical of the tumor and formed within the tumor or in a tissue adjacent to the tumor, said biomarkers being bound to ferromagnetic particles.

9. A contrast agent according to claim 8, wherein said biomarkers bind to a target molecule typical of a specific stage of a prostate tumor.

10. A contrast agent according to claim 9, comprising biomarkers that bind to the CEACAM-1 molecule.

11. A contrast agent according to claim 10, comprising a biomarker selected from the group consisting of antibodies, aptamers, spiegelmers, anticalins, viruses.

12. A contrast agent according to claim 9, wherein said biomarkers bind to VEGF.

13. A contrast agent according to claim 9, wherein said biomarkers bind to alpha(v)-beta(3)-integrin.

14. A contrast agent according to claim 12 comprising a biomarker selected from the group consisting of antibodies, ligands, aptamers, spiegelmers, anticalins, viruses.
